(19) United States
(12) Patent Application Publication
(10) Pub. No.: US 2019/0313925 A1
(43) Pub. Date: Oct. 17, 2019

(54) BLOOD PRESSURE MONITOR CUFF

(71) Applicant: GRANDWAY TECHNOLOGY (SHENZHEN) LIMITED, Shenzhen (CN)

(72) Inventor: Lai Yu CHOW, Kwun Tong (HK)

(73) Assignee: GRANDWAY TECHNOLOGY (SHENZHEN) LIMITED, Shenzhen (CN)

(21) Appl. No.: 16/204,068

(22) Filed: Nov. 29, 2018

(30) Foreign Application Priority Data
Apr. 12, 2018 (CN) 201820518695.6

(51) Int. Cl. A61B 5/022

(52) U.S. Cl. CPC A61B 5/02233; A61B 5/6831

(57) ABSTRACT

A blood pressure monitor cuff based on dial lacing system. Blood pressure monitor cuff on the market comprises rectangular or rectangular-like fabric that can secure blood pressure monitor at a certain position, in which fixing mechanism like Velcro tape is set on the fabric. Inconvenience due to limitations of the fixing mechanism, requires assistance of others such as a nurse. The present utility model could solve the above technical problems.

### FIELD

The present invention generally relates to accessories for diagnostic purposes and more particularly to a blood pressure monitor cuff.

### BACKGROUND

Blood pressure monitor cuff on the market comprises rectangular or rectangular-like fabric that can secure blood pressure monitor at a certain position, in which fixing mechanism like Velcro tape is set on the fabric. Such as an upper arm digital blood pressure monitor, when in use, wind fabric around the brachium and apply the mentioned fixing mechanism to secure the fabric around the brachium. Brachium is also known as upper arm.

When the upper arm digital blood pressure monitor is used, the cuff is secured around the upper arm. Limited by the range of motion of forearm, palm and fingers of the same arm are difficult or unable to reach the back of the upper arm, only the shoulder of the same arm could be reached. Since correct measurements can only be obtained when the cuff of digital blood pressure monitor is secured tightly around the arm, it is very difficult to secure and tighten the cuff at the same time no matter how both hands cooperate.

In order to solve this problem, some products provide a metal bar at one end of the cuff in which the width corresponds with the width of the cuff. Single operation of securing the cuffs could be achieved by passing one end of the cuff through the bar and pulling through the other end. However, the blood pressure monitor on the cuff might shift when pulling the cuff and causes incorrect measurement. When the user tries to adjust the position after securing the cuff, it would be difficult to readjust if the secure was tight, while the accuracy of measurement would be affected if the secure was loose.

Hence, the use of upper arm digital blood pressure monitor generally requires others, for instance assistance of nurse in the hospital.

The same technical problem exists in wrist digital blood pressure monitor as well; Due to the working principle of digital blood pressure monitor, above technical problems exist no matter the digital blood pressure monitor is separated or integrated with the cuff. While mercury blood pressure monitor is inconvenient to wear, it does not have shifting technical problem.

### SUMMARY

In order to solve the mentioned technical problem, the present invention reveals a kind of cuff that allows user to secure the cuff by himself/herself.

The blood pressure monitor cuff uses the lacing system based on a dial as the securing mechanism.

Specifically, said lacing system comprises at least one lace guide, in which one of the said lace guide and said dial is set in the middle of said cuff. Considering that the lace guide is available in a variety of shapes, at least one said lace guide is set on the opposite edge of cuff and said dial for ease of use.

The distance between said lace guide and said dial is equivalent to the circumference of an adult brachium.

In order to ensure the purpose of the invention could be achieved, the lace length of said lacing system should be greater or equal to 10 cm.

Considering when the cuff is not in use, the storage process may tangle the laces and cause inconvenience. So, lace opening of said lacing system is oriented at the outer part of cuff to minimize the possibility of tangling. This problem can also be solved through: said cuff comprises upper and lower layer, in which the lace of said lacing system is set in between upper and lower layer of said cuff. When applying this solution, the length of said cuff comprising upper- and lower-layer parts are at least 5 cm and above. Cuff consisting of lace support could further minimize the possibility of tangling.

To further facilitate the wear of cuff, the head and the end of said cuff is connected as a ring.

In addition to solving technical problems of inconvenient wear, above technical solutions can also solve shifting problem of cuff when said blood pressure monitor is digital blood pressure monitor.

### BRIEF DESCRIPTION OF THE DRAWINGS

FIGS. 1 and 2 is the securing of a cuff through a metal bar and Velcro tape as introduced in the background art.

FIG. 3 is the cuff in embodiment 1.

FIG. 4 is the operation diagram of in embodiment 2.

FIG. 5 is the cuff in embodiment 2.

FIG. 6 is the cuff after the upper part of the cuff is removed in embodiment 2.

FIG. 7 is the comparison before and after applying the dial to wind the cuff in embodiment 2.

FIG. 8 is the diagram of each components of the cuff in embodiment 2.

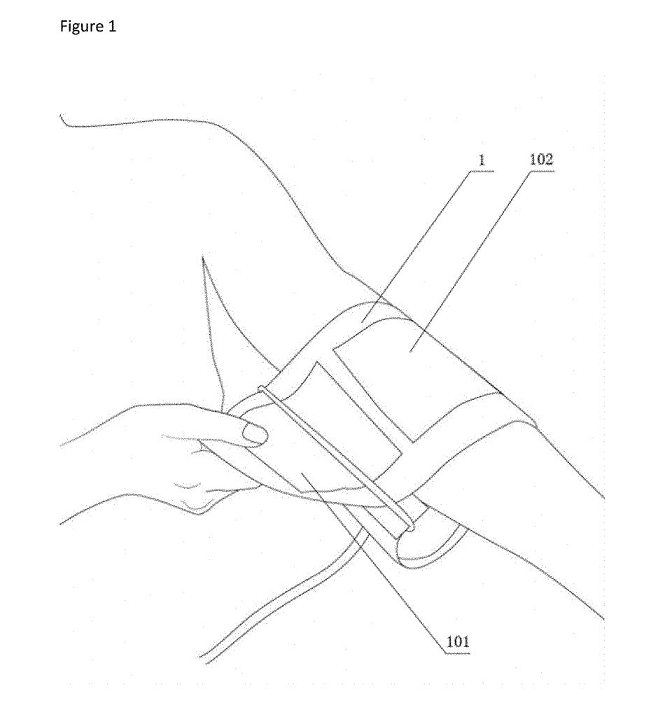

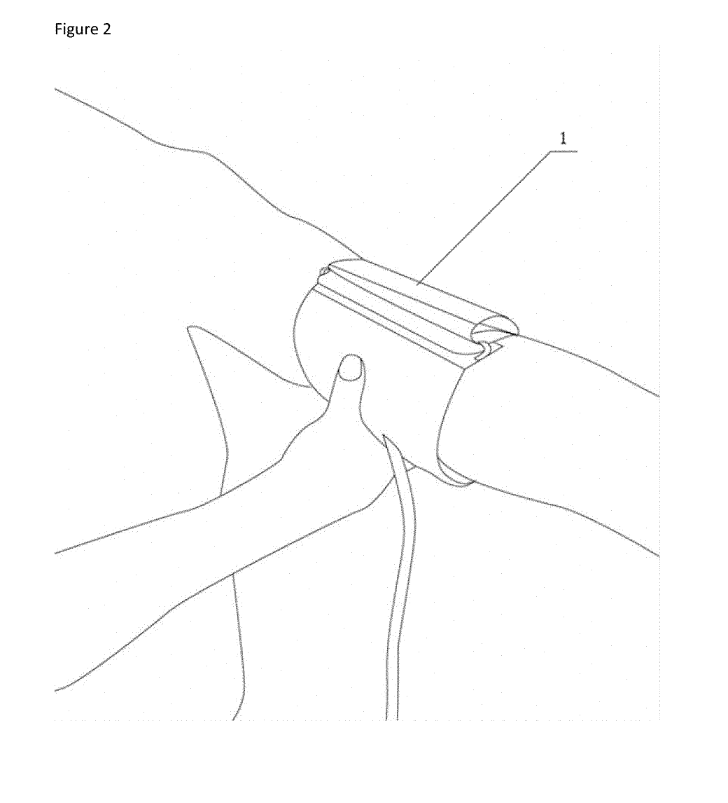

The numeral references in the figures are referred as: 1 is the cuff, 101 and 102 is the Velcro tape mentioned in the background art; 21 is the lace, 201 is the first lace guide in embodiment 1, 202 is the second lace guide in embodiment 2, 203 is the lace guide in embodiment 2, 204 is the dial. 3 is the airbag, 4 is the lace support.

### DETAILED DESCRIPTION

Boa Technology Inc. applied patents for lacing systems based on a dial in which include 201180032591.5. An embodiment will be applied to an upper arm blood pressure monitor using that lacing system as an example to solve technical problems mentioned in background art.

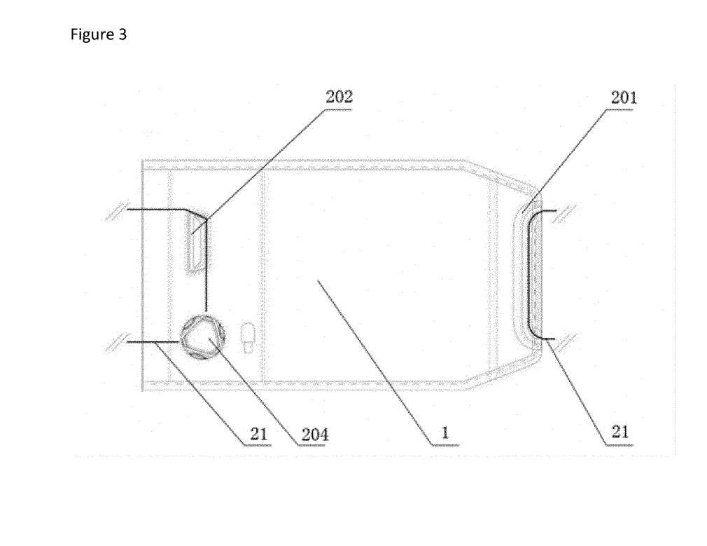

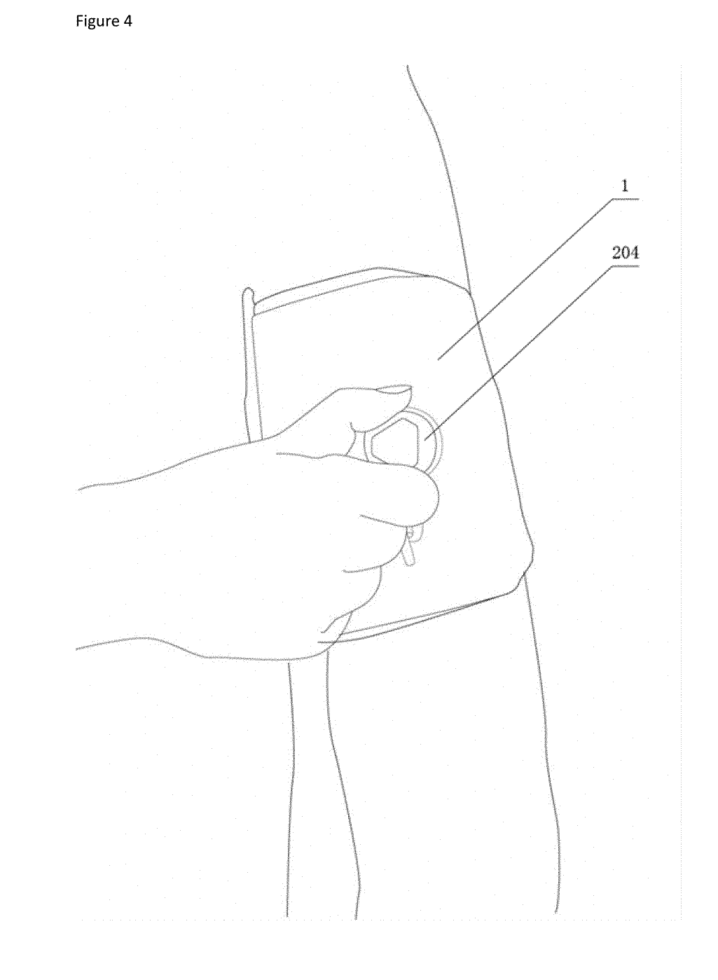

As shown in FIG. 3, cuff 1 is in a long strip shape. One of the edges consists of the first lace guide 201. In the embodiment, lace guide 201 is sewn on cuff 1 but it can also be fixed on the cuff through bonding, riveting and etc.

The middle of cuff 1 consist of the second lace guide 202 and dial 204. Lace 21 starts from dial 204, passes through the holes at both ends of the first lace guide 201, then passes through the holes at both ends of the second lace guide 202 and return to the dial.

By rotating dial 204, lace 21 can be winded so that the circumference of cuff 1 changes within a certain range. Considering the circumference of an adult upper arm, in this embodiment, the second lace guide 202 is in the same vertical position as the dial 204 and the distance between the first lace guide and the former two is the same. The distance between the first lace guide 201 and second lace guide 202 is 22 cm while the distance between the second lace guide 202 and the opposite edge is 27 cm. Thus, by rotating the dial 204, the circumference of the cuff 1 can be changed between 22 cm and 49 cm, so that the circumference of cuff 1 and an adult upper arm correspond with each other in order to tightly secure cuff 1 on the upper arm.

Since dial 204 is different from the pulling cuff and Velcro tape introduced in the background art, it does not produce a moment in the tangential direction of the upper arm, thus causing no twisting of the cuff which would further induce shifting of the blood pressure monitor, avoiding incorrect measurements caused by shifting of the blood pressure monitor.

One inconvenience of the embodiment shown in FIG. 3 is that the lace 21 is exposed. Tangling between lace and cuff or the exposed lace itself could happen during storage of the cuff, causing inconvenience in use.

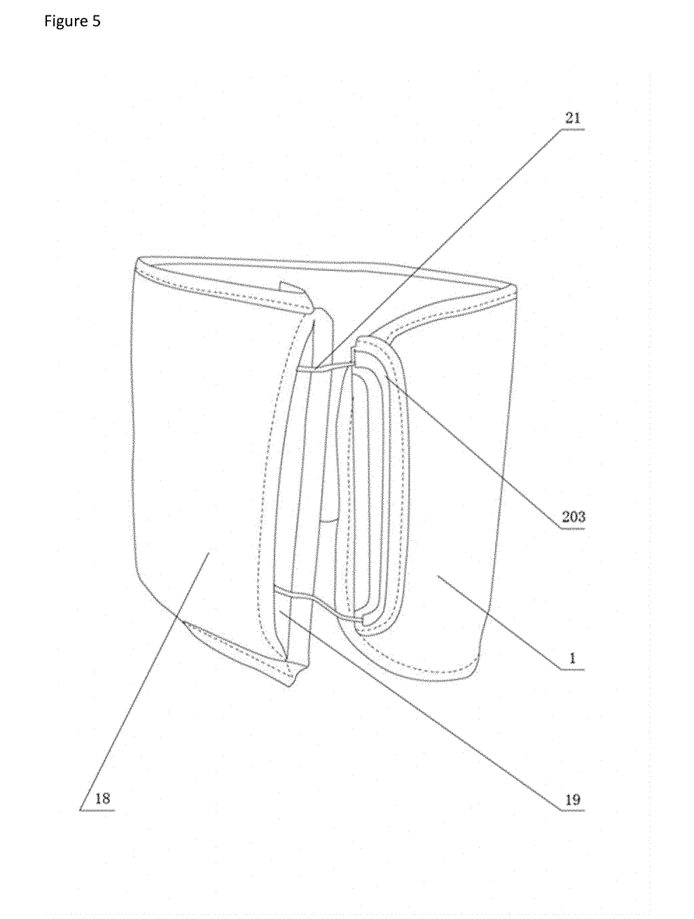

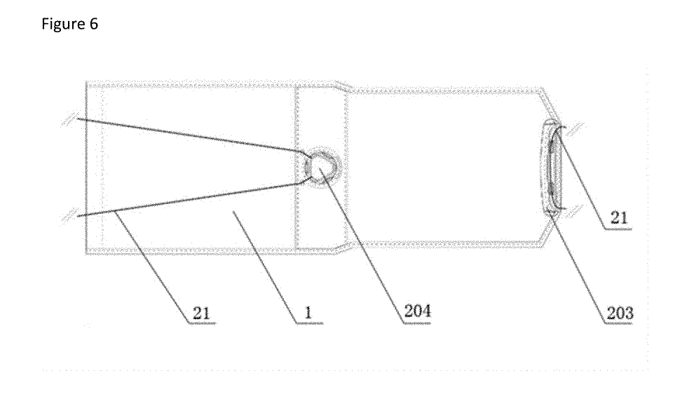

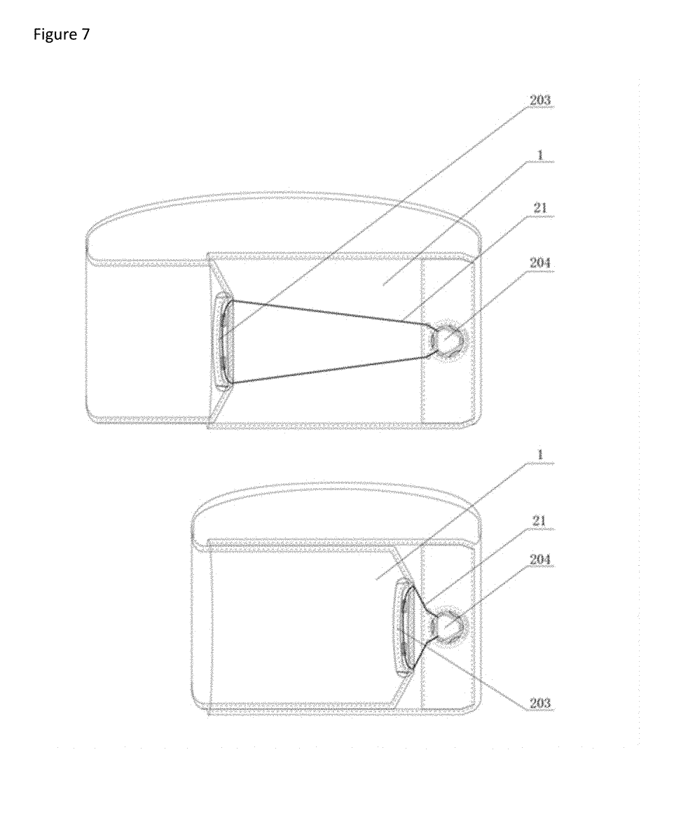

To this end, cuff 1 comprises upper layer cuff 18 and lower layer cuff 19 where lace 21 is set in between upper layer cuff 18 and lower layer cuff 19. The upper layer cuff 18 and lower layer cuff 19 are sewn or bonded to form a pathway in which lace 21 connects with dial 204 and lace guide 203 through the above two pathways, thereby forming embodiment 2, as shown in FIG. 5.

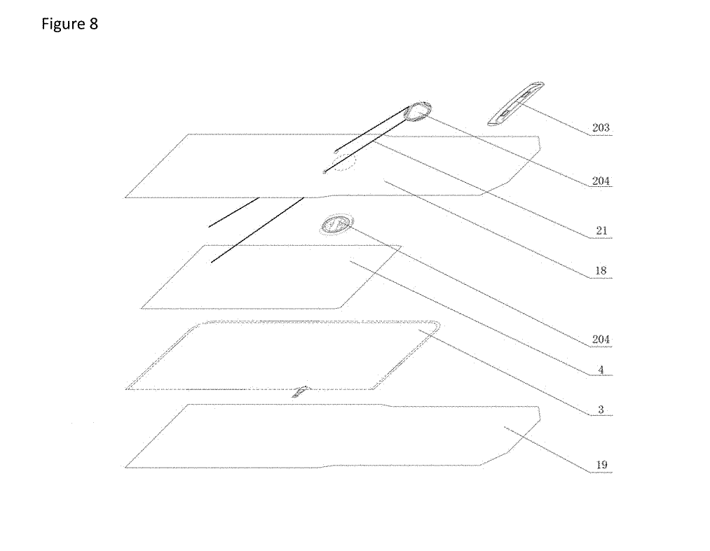

As shown in FIG. 8, in order to prevent lace 21 from shifting or tangling, lace support 4 is set in between upper layer cuff 18 and air bag 3. Lace support 4 can be made of components that have a certain stiffness such as a C-shaped rubber, supporting lace 21 through its own stiffness to restrict the degree of freedom, thereby reducing the possibility of shifting and tangling of lace 21.

The two ends of the cuff 1 are connected to each other through stitching, bonding and riveting so that cuff 1 is in a ring shape. When in use, cuff 1 can be first wrapped around the upper arm, then rotate the dial 204 to reduce the diameter of the cuff to an appropriate length, and further adjust the position of cuff to the correct measurement site. Further rotating dial 204 so that cuff 1 is secured around the user's upper arm. Considering the condition of the adult upper arm muscle, the diameter difference of cuff 1 during adjustment and after securing should be at least 1.5 cm and above, which is equivalent to circumference of approximately 5 cm and above. Thus, when the length of pathway is set above 5 cm, the length of lace is above 10 cm. Above operation could be completed entirely by user himself/herself, which is different from other prior technical solutions.

1. A blood pressure monitor cuff comprising lacing system based on a dial.

2. The blood pressure monitor cuff as claimed in claim 1, wherein said lacing system comprises at least one lace guide, in which one of the said lace guide and said dial is set in the middle of said cuff.

3. The blood pressure monitor cuff as claimed in claim 2, wherein at least one said lace guide is set on the opposite edge of cuff and said dial.

4. The blood pressure monitor cuff as claimed in claim 2, wherein the distance between said lace guide and said dial is equivalent to the circumference of an adult brachium.

5. The blood pressure monitor cuff as claimed in claim 2, wherein lace length of said lacing system is greater or equal to 10 cm.

6. The blood pressure monitor cuff as claimed in claim 2, wherein lace opening of said lacing system is oriented at the outer part of cuff.

7. The blood pressure monitor cuff as claimed in claim 1, wherein said cuff comprises upper and lower layer, in which the lace of said lacing system is set in between upper and lower layer of said cuff.

8. The blood pressure monitor cuff as claimed in claim 7, wherein said cuff comprises upper- and lower-layer parts with length 5 cm and above.

9. The blood pressure monitor cuff as claimed in claim 7, wherein said cuff comprises lace support.

10. The blood pressure monitor cuff as claimed in claim 1, wherein said blood pressure monitor is digital blood pressure monitor.
